(19) United States
(12) Patent Application Publication
(10) Pub. No.: US 2014/0051840 A1
(43) Pub. Date: Feb. 20, 2014

(54) ARSENIC COMPLEXES FOR POTENTIAL DIAGNOSTIC APPLICATIONS

(71) Applicant: The Curators of the University of Missouri, Columbia, MO (US)

(72) Inventors: Silvia S. Jurisson, Columbia, MO (US); Cathy S. Cutler, Columbia, MO (US); Donald E. Wycoff, Columbia, MO (US); Anthony J. DeGraffenreid, Columbia, MO (US)

(21) Appl. No.: 13/969,196

(22) Filed: Aug. 16, 2013

Related U.S. Application Data
(60) Provisional application No. 61/684,421, filed on Aug. 17, 2012.

(51) Int. Cl. A61K 51/04; A61K 51/10; A61K 51/08

(52) U.S. Cl. CPC A61K 51/0474; A61K 51/08; A61K 51/10
USPC 530/391.3; 534/10; 530/327

(57) ABSTRACT

The present invention provides radioactive arsenic complex useful in diagnostic and therapeutic applications and methods for forming those arsenic complexes.

### CROSS-REFERENCE

This application claims priority to U.S. Ser. No. 61/684,421 filed Aug. 17, 2012, which is incorporated herein by reference in its entirety.

### STATEMENT REGARDING FEDERAL RIGHTS

This invention was made with government support under Grant No. DE-SC0003851 awarded by the United States Department of Energy, and under Training Grant No. ST32-EB004822 awarded by the National Institute of Biomedical Imaging and Bioengineering (NIBIB) at the National Institutes of Health (NIH), an agency of the United States Department of Health and Human Services. The government has certain rights to the invention.

### FIELD OF THE INVENTION

The present invention generally relates to arsenic complexes, especially to radioactive arsenic complexes useful in diagnostic and therapeutic applications and to methods for forming those complexes.

### BACKGROUND OF THE INVENTION

Currently, positron emission tomography (PET) imaging relies upon ¹⁸F as the positron emitter, typically in the form of fluorodeoxyglucose (FDG), which is metabolized by active body tissue. With a short half-life of only 1.8 hours, radiopharmaceuticals containing ¹⁸F must be synthesized immediately before administration to patients near the cyclotron source of the element. Such a short half-life does not permit sufficient time to radiolabel peptides and antibodies and does not permit these radiopharmaceuticals to be used in radiotherapy.

Because of their significantly longer half-life than ¹⁸F, arsenic radioisotopes permit a much wider range of radiopharmaceuticals, including labeling biological vectors. As such, radiotherapeutic agents can then be developed for targeting a variety of diseases using arsenic radiolabeled biomolecules, adding to the arsenal of potential cancer therapeutics.

What is needed then are more reliable methods for preparing arsenic radioisotopes and compounds containing those radioisotopes.

### SUMMARY OF THE INVENTION

Briefly, therefore, one aspect of the present disclosure encompasses processes for preparing radiolabeled phenylarsonic acid, C₆H₅AsO₃H₂, from radiolabeled arsenous acid, either as As₂O₃ or as H₃AsO₃. The phenylarsonic acid may be used as a precursor for radiolabeled arsenic complexes for diagnostic or therapeutic nuclear medicine. These arsenic complexes are formed by reaction between the radiolabeled phenylarsenic acid and a sulfur-containing reagent, such as a thioamine, monothiol, or a dithiol.

Another aspect of invention, the disclosure provides a composition comprising a compound of Formula (I):

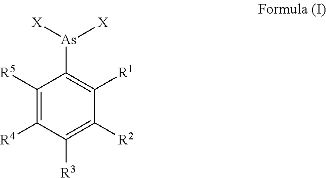

wherein arsenic is radioactive;

R¹, R², R³, R⁴, and R⁵ are independently selected from hydrogen, hydrocarbyl, substituted hydrocarbyl, amines, substituted amines, halogens, hydroxyl, thiol, or nitro groups or are joined together to form one or more fused ring systems selected from hydrocarbyl, substituted hydrocarbyl, aromatic, and heteroaromatic rings;

X represents ligand atoms wherein X is chosen from carbon, sulfur, and nitrogen;

X may be further substituted by hydrogen, hydrocarbyl or substituted hydrocarbyl.

In another aspect of the disclosure, there is provided a composition comprising Formula (II):

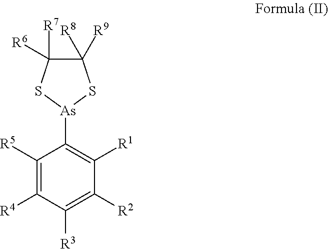

wherein arsenic is radioactive;

R¹, R², R³, R⁴, and R⁵ are independently selected from hydrogen, hydrocarbyl, substituted hydrocarbyl, amines, substituted amines, halogens, hydroxyl, thiol, or nitro groups or are joined together to form one or more fused ring systems selected from hydrocarbyl, substituted hydrocarbyl, aromatic and heteroaromatic rings; and

R⁶, R⁷, R⁸, and R⁹ are independently selected from hydrogen, hydrocarbyl, substituted hydrocarbyl, amines, substituted amines, halogens, hydroxyl, thiol, or nitro groups or are joined together to form one or more fused ring systems selected from hydrocarbyl and substituted hydrocarbyl rings and aromatic and heteroaromatic rings.

In yet another aspect of the disclosure, there is provided a composition comprising Formula (III):

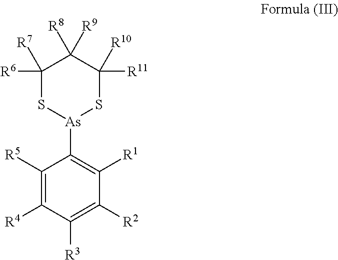

wherein arsenic is radioactive;

R¹, R², R³, R⁴, and R⁵ are independently selected from hydrogen, hydrocarbyl, substituted hydrocarbyl, amines, substituted amines, halogens, hydroxyl, thiol, or nitro groups or are joined together to form one or more fused ring systems selected from hydrocarbyl, substituted hydrocarbyl rings, aromatic and heteroaromatic rings; and

R⁶, R⁷, R⁸, R⁹, R¹⁰, and R¹¹ are independently selected from hydrogen, hydrocarbyl, substituted hydrocarbyl, amines, substituted amines, halogens, hydroxyl, thiol, or nitro groups or are joined together to form one or more fused ring systems selected from hydrocarbyl, substituted hydrocarbyl, aromatic and heteroaromatic rings.

In other aspects of the disclosure, there is provided a radiopharmaceutical comprising Formula (I), (II), or (III):

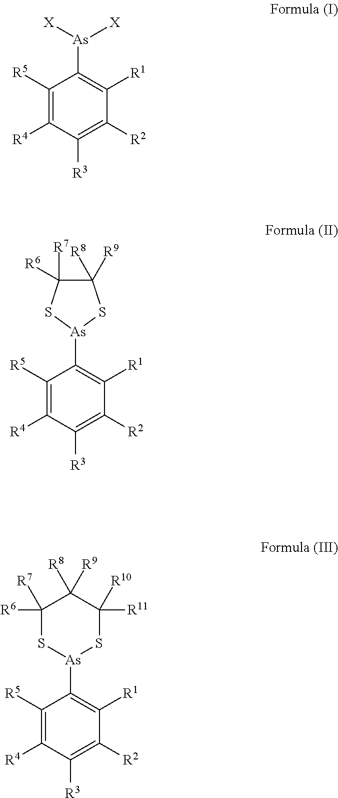

wherein arsenic is radioactive;

X represents ligand atoms wherein X is chosen from carbon, sulfur, and nitrogen;

X may be further substituted with hydrogen, hydrocarbyl or substituted hydrocarbyl;

R¹, R², R³, R⁴, and R⁵ are independently selected from hydrogen, hydrocarbyl, substituted hydrocarbyl, amines, substituted amines, halogens, hydroxyl, thiol, or nitro groups or are joined together to form one or more fused ring systems selected from hydrocarbyl, substituted hydrocarbyl, aromatic and heteroaromatic rings; and

R⁶, R⁷, R⁸, R⁹, R¹⁰, and R¹¹ are independently selected from hydrogen, hydrocarbyl, substituted hydrocarbyl, amines, substituted amines, halogens, hydroxyl, thiol, or nitro groups or are joined together to form one or more fused ring systems selected from hydrocarbyl and substituted hydrocarbyl rings and aromatic and heteroaromatic rings.

In still other aspects of the disclosure, there is provided a method for producing radiolabeled phenylarsonic acid. The method comprises contacting a labeled arsenous acid with a carbonate to form a first solution; (b) optionally contacting the first solution with a copper-based catalyst to form a second solution; and (c) contacting the second solution with a phenyldiazonium salt to give a third solution comprising the radiolabeled phenylarsonic acid.

Other features and iterations of the invention are described in more detail below.

### DETAILED DESCRIPTION OF THE INVENTION

The present invention provides arsenic complexes containing radioactive arsenic as well as processes for preparing those arsenic complexes.

### (I) Compounds

In general, the disclosure provides a composition comprising a compound of Formula (I):

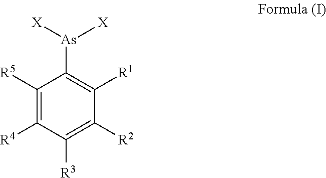

wherein arsenic is radioactive;

R¹, R², R³, R⁴, and R⁵ are independently selected from hydrogen, hydrocarbyl, substituted hydrocarbyl, amines, substituted amines, halogens, hydroxyl, thiol, or nitro groups or are joined together to form one or more fused ring systems selected from hydrocarbyl, substituted hydrocarbyl, aromatic, and heteroaromatic rings;

X represents ligand atoms wherein X is chosen from carbon, sulfur, and nitrogen;

X may be further substituted by hydrogen, hydrocarbyl or substituted hydrocarbyl.

In some embodiments, the arsenic may be chosen from ⁷²As or ⁷⁷As. In other embodiments, the R³ may be NH₂. In still other embodiments, R³ may be NH₂, and R¹, R², R⁴, and R⁵ are hydrogen. In some iterations, each X may be sulfur and may be substituted by hydrogen, hydrocarbyl or substituted hydrocarbyl. In other iterations, one or both X may be substituted by carboxylic acids, phenyl groups or amines. In still other iterations, one or both X may be substituted by CH₂COOH. In some other iterations, one or both X may be substituted by CH₂Ph. In other iterations, one or both X may be substituted by CH₂CH₂NH₂. In some embodiments, the composition may be isolated.

In another aspect of the disclosure, there is provided a composition comprising Formula (II):

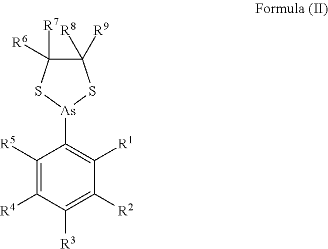

wherein arsenic is radioactive;

R¹, R², R³, R⁴, and R⁵ are independently selected from hydrogen, hydrocarbyl, substituted hydrocarbyl, amines, substituted amines, halogens, hydroxyl, thiol, or nitro groups or are joined together to form one or more fused ring systems selected from hydrocarbyl, substituted hydrocarbyl, aromatic and heteroaromatic rings; and

R⁶, R⁷, R⁸, and R⁹ are independently selected from hydrogen, hydrocarbyl, substituted hydrocarbyl, amines, substituted amines, halogens, hydroxyl, thiol, or nitro groups or are joined together to form one or more fused ring systems selected from hydrocarbyl and substituted hydrocarbyl rings and aromatic and heteroaromatic rings.

In some embodiments, the arsenic may be chosen from ⁷²As or ⁷⁷As. In other embodiments, R³ may be NH₂, and R¹, R², R⁴, and R⁵ may each be hydrogen. In still other embodiments, R⁶, R⁷, R⁸, and R⁹ may each be hydrogen. In some iterations, one or more of R⁶, R⁷, R⁸, and R⁹ may be substituted with a C₁-C₁₀ carboxylic acid. In other iterations, R⁶ and R⁸ may be substituted by COOH. In some other iterations, R⁷ and R⁹ may be substituted by hydrogen. In still other iterations, one of R⁶, R⁷, R⁸, or R⁹ may be substituted by a linear C₁-C₅ carboxylic acid. In some embodiments, R⁶, R⁷, R⁸, or R⁹ may be substituted such to form a fused ring system with one or more hydrocarbyl, substituted hydrocarbyl, aromatic or heteroaromatic rings. In other embodiments, the composition may be isolated.

In yet another aspect of the disclosure, there is provided a composition comprising Formula (III):

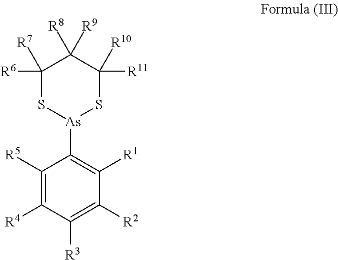

wherein arsenic is radioactive;

R¹, R², R³, R⁴, and R⁵ are independently selected from hydrogen, hydrocarbyl, substituted hydrocarbyl, amines, substituted amines, halogens, hydroxyl, thiol, or nitro groups or are joined together to form one or more fused ring systems selected from hydrocarbyl, substituted hydrocarbyl rings, aromatic and heteroaromatic rings; and

R⁶, R⁷, R⁸, R⁹, R¹⁰, and R¹¹ are independently selected from hydrogen, hydrocarbyl, substituted hydrocarbyl, amines, substituted amines, halogens, hydroxyl, thiol, or nitro groups or are joined together to form one or more fused ring systems selected from hydrocarbyl, substituted hydrocarbyl, aromatic and heteroaromatic rings.

In some embodiments, the arsenic may be chosen from ⁷²As or ⁷⁷As. In other embodiments, R³ may be NH₂, and R¹, R², R⁴, and R⁵ may each be hydrogen. In some iterations, R⁶, R⁷, R⁸, R⁹, R¹⁰, and R¹¹ may each be hydrogen. In other iterations, one or more of R⁶, R⁷, R⁸, R⁹, R¹⁰, and R¹¹ may be substituted with a C₁-C₁₀ carboxylic acid. In some other iterations, one or more of R⁶, R⁸, or R¹⁰ may be substituted by COOH. In still other iterations, one of R⁶, R⁷, R⁸, R⁹, R¹⁰, and R¹¹ may be substituted by a C₁-C₅ carboxylic acid. In some embodiments, two or more of R⁶, R⁷, R⁸, R⁹, R¹⁰, and R¹¹ may be substituted such to form a fused ring system with one or more hydrocarbyl, substituted hydrocarbyl, aromatic or heteroaromatic ring systems. In other embodiments, the composition may be isolated.

In other aspects of the disclosure, there is provided a radiopharmaceutical comprising Formula (I), (II), or (III):

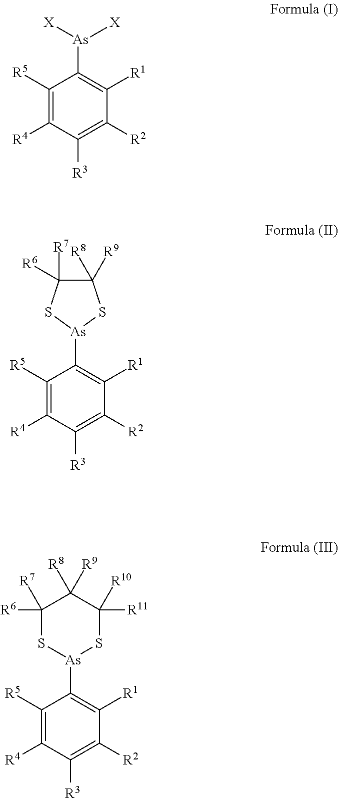

wherein arsenic is radioactive;

X represents ligand atoms wherein X is chosen from carbon, sulfur, and nitrogen;

X may be further substituted with hydrogen, hydrocarbyl or substituted hydrocarbyl;

R¹, R², R³, R⁴, and R⁵ are independently selected from hydrogen, hydrocarbyl, substituted hydrocarbyl, amines, substituted amines, halogens, hydroxyl, thiol, or nitro groups or are joined together to form one or more fused ring systems selected from hydrocarbyl, substituted hydrocarbyl, aromatic and heteroaromatic rings; and

R⁶, R⁷, R⁸, R⁹, R¹⁰, and R¹¹ are independently selected from hydrogen, hydrocarbyl, substituted hydrocarbyl, amines, substituted amines, halogens, hydroxyl, thiol, or nitro groups or are joined together to form one or more fused ring systems selected from hydrocarbyl and substituted hydrocarbyl rings and aromatic and heteroaromatic rings.

In some embodiments, the arsenic may be chosen from ⁷²As or ⁷⁷As. In other embodiments, R³ may be NH₂, and R¹, R², R⁴, and R⁵ may each be hydrogen. In some iterations, R⁶, R⁷, R⁸, R⁹, R¹⁰, and R¹¹ may be independently selected from hydrogen, hydrocarbyl, and substituted hydrocarbyl. In other iterations, R⁶, R⁷, R⁸, R⁹, R¹⁰, and R¹¹ may be independently selected from hydrogen, carboxylic acid, and C₁-C₁₀ carboxylic acids.

In an exemplary embodiment, the compound of Formula (I), (II), or (III) may be conjugated to a peptide. The peptide may be, for example, bombesin or a site-specific antibody.

### (a) Radioactive Arsenic

The compounds of Formulas (I), (II), and (III) contain radioactive arsenic. Radioactive isotopes of arsenic include ⁶⁰As, ⁶¹As, ⁶²As, ⁶³As, ⁶⁴As, ⁶⁵As, ⁶⁶As, ^{66m1}As, ^{66m2}As, ⁶⁷As, ⁶⁸As, ^{68m}As, ⁶⁹As, ⁷⁰As, ^{70m}As, ⁷¹As, ⁷²As, ⁷³As, ⁷⁴As, ^{75m}As, ⁷⁶As, ^{76m}As, ⁷⁷As, ^{77m}As, ⁷⁸As, ⁷⁹As, ^{79m}As, ⁸⁰As, ⁸¹As, ⁸²As, ^{82m}As, ⁸³As, ⁸⁴As, ^{84m}As, ⁸⁵As, ⁸⁶As, ⁸⁷As, ⁸⁸As, ⁸⁹As, ⁹⁰As, ⁹¹As, and ⁹²As. In some embodiments, the arsenic isotope may be relatively long-lived. For example, the isotope may have a half-life of at least one day. Examples of relatively long-lived radioactive isotopes of arsenic include ⁶⁸As, ⁶⁹As, ⁷⁰As, ⁷¹As, ⁷²As, ⁷³As, ⁷⁴As, ⁷⁶As, ⁷⁷As, ⁷⁸As, and ⁷⁹As. In exemplary embodiments, the radioisotope of arsenic may be ⁷²As or ⁷⁷As.

Arsenic radioisotopes may be selected for their desirable nuclear properties, such as a half-life that is sufficiently long to permit chemical derivation, radiodiagnostic imaging, and/or radiotherapy, but short enough that the radionuclide can clear the patient's body within a suitable time after administration. Arsenic radioisotopes may also be selected that undergo a high percentage of beta decay, rather than other modes of radioactive decay, such as electron capture (EC). A high percentage of positron (β⁺) decay is desirable for positron emission tomography (PET), which relies upon positron emission of the radioisotope for imaging, and a high percentage of gamma emission for single-photon emission computed tomography (SPECT) imaging. Another desirable property of arsenic radioisotopes is a maximum energy for positron emission (Eβₘₐₓ) that permits image resolution similar to images acquired from an ¹⁸F-labeled compound. The properties for some exemplary arsenic radioisotopes are shown below in Table 1:

TABLE 1
Properties of exemplary arsenic radioisotopes

| Property | ⁷⁰As | ⁷¹As | ⁷²As | ⁷⁴As | ⁷⁶As |
|---|---|---|---|---|---|
| Half-life | 52.5 min | 2.7 days | 1.1 days | 17.8 days | 1.6 days |
| (t_{1/2}) | | | | | |
| Mode of | β⁺ | EC (68%); | β⁺ (87.8%); | EC (66%); | β⁻ (100%) |
| decay (%) | (100%) | β⁺ (32%) | EC (12.2%) | β⁺ (29%) | |
| Eβₘₐₓ | 1.44 | 2.01 | 2.5 | 0.94 | 2.94 |
| (MeV) | | | | | |

* EC = electron capture

### (b) Biological Vectors

In vivo stable compounds of radiolabeled arsenic complexes linked to biological vectors enable a new class of PET imaging agents to be developed for a wide range of medical applications. For example, the half-life of ⁷²As is 26 hours, permitting its use as a label for peptides and possibly antibodies giving specificity in target imaging. In another example, in vivo stable compounds of ⁷⁷As linked to biological vectors would enable a new class of radiotherapeutic agents to be developed for a wide range of medical applications. For instance, ⁷⁷As would allow simultaneous radiotherapy and SPECT imaging by emission and detection of 239-keV gamma radiation. In other examples, the annihilation of emitted positrons from a radioactive arsenic species may produce gamma radiation which can be detected through SPECT imaging.

In some embodiments, the biological vector may be a peptide. In various embodiments, the peptide has a relatively low molecular weight and rapid in vivo blood clearance. Non-limiting examples of peptides include bombesin, site-directed antibodies, neuromedin B, neuromedin N, neuromedin S, neuromedin U; angiotensin, calcitonin gene-related peptide, carnosine, cocaine and amphetamine regulated transcript, delta sleep-inducing peptide, FMRFamide (Phe-Met-Arg-Phe), galanin, galanin-like peptide, gastrin-releasing peptide, neuropeptide S, neuropeptide Y, neurophysins, neurotensin, pancreatic polypeptide, pituitary adenylated cyclase-activating peptide, RVD-Hpa, VGF; a kinin, such as cholecystokinin, bradykinin or tachykinin, for example substance P, neurokinin A, neurokinin B, kassinin, or physalaemin; a dynorphin, such as dynorphin A or dynorphin B; an endorphin, such as beta-endorphin, alpha-endorphin, gamma-endorphin, α-neo-endorphin, or β-neo-endorphin; an encephalin, such as Met-encephalin or Leu-encephalin; adrenorphin, amidorphin, leumorphin, nociception, oriorphin, spinorphin; or a peptide hormone, such as prolactin, adrenocorticotropic hormone (ACTH), growth hormone, vasopressin, oxytocin, atrial-natriuretic peptide (ANP), atrial-natriuretic factor (ANF), glucagon, insulin, somatostatin, or leptin. In an exemplary embodiment, the peptide bombesin, a 14-amino acid peptide originally isolated from the skin of the oriental fire-bellied toad (Bombina orientalis). Bombesin stimulates gastrin release from G cells, in particular activating the G-protein-coupled receptors BBR1, BBR2, and BBR3. In other exemplary embodiments, the peptide is a site-directed antibody; that is, an immunoglobulin designed to recognize specific targets.

(c) Downstream applications

In some embodiments, the compound comprising Formula (I), (II) or (III) may be converted into a pharmaceutically acceptable salt. “Pharmaceutically-acceptable salts” are salts commonly used to form alkali metal salts and to form addition salts of free acids or free bases. The nature of the salt may vary, provided that it is pharmaceutically acceptable. Suitable pharmaceutically acceptable acid addition salts of compounds comprising Formula (I), (II), or (III) may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, hydroxybutyric, salicylic, galactaric and galacturonic acid. Suitable pharmaceutically-acceptable base addition salts include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine-(N-methylglucamine) and procaine. All of these salts may be prepared by conventional means from the corresponding compound by reacting, for example, the appropriate acid or base with the any compound comprising Formula (I), (II), or (III).

### (d) Stereochemistry

The compound comprising any of Formulas (I), (II), or (III) may have a (−) or a (+) orientation with respect to the rotation of polarized light. More specifically, each chiral center of the arsenic complex may have an R or an S configuration.

### (II) Process for Preparation of Arsenic Complexes

In still other aspects of the disclosure, there is provided a method of producing radiolabeled phenylarsonic acid. The method comprises contacting a radiolabeled arsenous acid with a carbonate to form a first solution. The first solution may optionally be contacted with a copper-based catalyst to form a second solution. The second solution is contacted with a phenyldiazonium salt to give a third solution comprising the labeled phenylarsonic acid. When a copper-based catalyst is not used, the first and second solutions are the same.

In some embodiments, the radiolabeled arsenous acid may be chosen from As₂O₃ or H₃AsO₃. In an exemplary embodiment, the carbonate may be sodium carbonate. In other embodiments, the phenyldiazonium salt may be a tetrafluoroborate salt. In some embodiments, the first solution may be heated to about 70° C. to about 90° C. In other embodiments, the copper-based catalyst may be copper(II) sulfate (CuSO₄), and the mole to mole ratio of arsenous acid to CuSO₄ may range from about 1:2 to 1:3 arsenous acid to CuSO₄. In an exemplary embodiment, the contact between the second solution and the phenyldiazonium salt may be conducted at about 0° C. In another exemplary embodiment, the process may further comprise neutralizing and filtering the third solution. In other embodiments, the radiolabeled phenylarsonic acid may be isolated. In an exemplary embodiment, the phenylarsonic acid may be further reacted with a thioamine and a monothiol or dithiol.

### (a) Step A—Reaction Mixture

(i) Radiolabeled Arsenous Acid

The arsenous acid may be in any form, for example as arsenic trioxide (As₂O₃) or in a form that has equilibrated with water (H₃AsO₃). The arsenous acid may be radiolabeled with any radioactive arsenic described above in section (I)(a).

(ii) Carbonate

The arsenous acid is contacted with carbonate. Non-limiting examples of carbonate include sodium carbonate, potassium carbonate, lithium carbonate, and magnesium carbonate. In an exemplary embodiment, the carbonate is sodium carbonate.

The amount of carbonate added to the reaction mixture can and will vary. In general, the mole:mole ratio of the arsenous acid to the carbonate may range from about 1:5 to about 1:500. In various embodiments, the molar ratio of arsenous acid to the carbonate may range from about 1:5 to about 1:10, from about 1:10 to about 1:50, from about 1:50 to about 1:100, from about 1:100 to about 1:200, from about 1:200 to about 1:300, from about 1:300 to about 1:400, or from about 1:400 to about 1:500. In one exemplary embodiment, the mole:mole ratio of the arsenous acid to carbonate may range from about 1:40 to about 1:60.

(iii) Solvent

The reaction is generally conducted in the presence of a solvent or solvent system. The solvent may be a polar protic solvent, a polar aprotic solvent, or a nonpolar organic solvent. Non-limiting examples of suitable polar protic solvents include methanol, ethanol, isopropanol, n-propanol, isobutanol, n-butanol, s-butanol, t-butanol, formic acid, acetic acid, water, and combinations thereof. Non-limiting examples of suitable polar aprotic solvents include acetone, acetonitrile, diethoxymethane, N,N-dimethylformamide (DMF), dimethyl sulfoxide (DMSO), N,N-dimethylpropanamide (or dimethylpropionamide; DMP), 1,3-dimethyl-3,4,5,6-tetrahydro-2(1H)-pyrimidinone (DMPU), 1,3-dimethyl-2-imidazolidinone (DMI), 1,2-dimethoxyethane (DME), dimethoxymethane, bis(2-methoxyethyl)ether, N,N-dimethylacetamide (DMA), N-methyl-2-pyrrolidinone (NMP), 1,4-dioxane, ethyl formate, formamide, hexachloroacetone, hexamethylphosphoramide, methyl acetate, N-methylacetamide, N-methylformamide, methylene chloride, methoxyethane, morpholine, nitrobenzene, nitromethane, propionitrile, pyridine, sulfolane, tetramethylurea, tetrahydrofuran (THF), 2-methyl tetrahydrofuran, tetrahydropyran, trichloromethane, and combinations thereof. Representative nonpolar solvents include, but are not limited to, alkane and substituted alkane solvents (including cycloalkanes), aromatic hydrocarbons, esters, ethers, ketones, and combinations thereof. Specific nonpolar solvents that may be employed include, for example, benzene, butyl acetate, t-butyl methylether, t-butyl methylketone, chlorobenzene, chloroform, cyclohexane, dichloromethane, dichloroethane, diethyl ether, ethyl acetate, fluorobenzene, heptane, hexanes, methyl ethylketone (2-butanone), methyl isobutyl ketone, pentyl acetate, propyl acetates, toluene, and combinations thereof. In an exemplary embodiment, the solvent is a polar protic solvent. In particular the solvent is water, deuterium oxide (D₂O), or combinations thereof.

In general, the volume to mass ratio of the solvent to arsenous acid may range from about 0.001:1 to about 1:1. In various embodiments, the volume to mass ratio of the solvent to arsenous acid may range from 0.001:1 to about 0.01:1, from about 0.01:1 to about 0.1:1, or from about 0.1:1 to about 1:1. In exemplary embodiments, the volume to mass ratio of the solvent to arsenous acid may range from about 0.01:1 to about 0.05:1.

### (b) Step A—Reaction Conditions

In general, the reaction may be conducted at a temperature that ranges from about 0° C. to about 120° C. In various embodiments, the reaction may be conducted at a temperature from about 0° C. to about 20° C., from about 20° C. to about 40° C., from about 40° C. to about 60° C., from about 60° C. to about 80° C., from about 80° C. to about 100° C., or from about 100° C. to about 120° C. In an exemplary embodiment, the reaction may be conducted at a temperature of about 70° C. to about 90° C. The reaction generally is conducted in an inert atmosphere (e.g., under nitrogen or argon) and under ambient pressure.

Typically, the reaction is allowed to proceed for a sufficient period of time until the reaction is complete, as determined by chromatography (e.g., HPLC) or another suitable method. In this context, a “completed reaction” generally means that the reaction mixture contains a significantly diminished amount of arsenous acid, and a significantly increased amount of deprotonated arsenous acid compared to the amounts of each present at the beginning of the reaction. In general, the reaction may proceed for about 1 minute to about 12 hours. Typically, the duration of the reaction is longer at lower reaction temperatures. In certain embodiments, the reaction may be allowed to proceed for a period of time ranging from about 1 minute to about 5 minutes, from about 5 minutes to about 10 minutes, from about 10 minutes to about 15 minutes, from about 15 minutes to about 30 minutes, from about 30 minutes to about 1 hour, from about 1 hour to about 3 hours, from about 3 hours to about 6 hours, from about 6 hours to about 9 hours, or from about 9 hours to about 12 hours.

In some embodiments, the deprotonated arsenous acid may be isolated from the reaction mixture using techniques known to those of skill in the art. Non-limiting examples of suitable techniques include precipitation, extraction, evaporation, distillation, chromatography, and crystallization. In other embodiments, the deprotonated arsenous acid may not be isolated and step (b) of the process may proceed in the same reaction pot or reactor.

The yield of the deprotonated arsenous acid can and will vary. Typically, the yield of the deprotonated arsenous acid may be at least about 40%. In one embodiment, the yield of the deprotonated arsenous acid may range from about 40% to about 60%. In another embodiment, the yield of the deprotonated arsenous acid may range from about 60% to about 80%. In a further embodiment, the yield of the deprotonated arsenous acid may range from about 80% to about 90%. In still another embodiment, the yield of the deprotonated arsenous acid may be greater than about 90%, or greater than about 95%.

### (c) Step B—Reaction Mixture

The method for producing a radiolabeled phenylarsonic acid may further comprise contacting the solution from Step A with a copper-based catalyst. The copper atom in the copper-based catalyst may have an oxidation state of 0, 1+, 2+, or 3+. Non-limiting examples of suitable copper-based catalysts include copper(I) bromide (CuBr), copper(I) cyanide (CuCN), copper(I) chloride (CuCl), copper(I) fluoride (CuF), copper(I) iodide (CuI), copper chromite (Cu₂Cr₂O₅), copper(I) oxide (Cu₂O), copper(I) sulfide (Cu₂S), copper(I) phosphate (Cu₃(PO₄)₂), copper(I) phosphide (Cu₃P), copper tetrafluoroborate (Cu(BF₄)₂), copper(II) bromide (CuBr₂), copper(II) carbide (CuC₂), copper(II) cyanide (Cu(CN)₂), copper(II) carbonate (CuCO₃), copper(II) chloride (CuCl₂), copper(II) fluoride (CuF₂), copper(II) nitrate (Cu(NO₃)₂), copper(II) oxide (CuO), copper(II) hydroxide (Cu(OH)₂), copper(II) iodide (CuI₂), copper(II) sulfide (CuS), copper(II) sulfate (CuSO₄), copper(II) arsenate (Cu₃(AsO₄)₂), potassium hexafluorocuprate (K₃CuF₆), copper(III) oxide (Cu₂O₃), and cesium hexafluorocuprate (Cs₂CuF₆). In an exemplary embodiment, the copper-based catalyst may be copper(II) sulfate (CuSO₄).

In some embodiments, the copper-based catalyst may be the copper metal element itself. For example, the copper may be a powder or a sponge. In other embodiments, the copper may be immobilized on a solid surface or support. The copper-based catalyst may be soluble (i.e., homogeneous) or may be immobilized on a solid support (i.e., heterogeneous), for example via noncovalent or covalent bonds. In some embodiments, the solid support may be an inorganic material. Suitable inorganic materials include silicas, alumina, titania, carbondium, zirconia, activated charcoal, zeolites, clays, polymers, ceramics, and activated carbon. Suitable silicas include silicon dioxide, amorphous silica, and microporous or mesoporous silicas. In other embodiments, the solid support may be a polymer. The polymer may be a natural polymer, a synthetic polymer, a semi-synthetic polymer, or a copolymer. Non-limiting examples of polymers include agarose, cellulose, nitrocellulose, methyl cellulose, polyacrylic, polyacrylamide, polyacrylonitrile, polyamide, polyether, polyester, polyethylene, polystyrene, polysulfone, polyvinyl chloride, polyvinylidene, methacrylate copolymer, and polystyrene-vinyl chloride copolymer.

The amount of copper-based catalyst added to the reaction mixture can and will vary. In general, the mole:mole ratio of the deprotonated arsenous acid to the copper-based catalyst may range from about 1:0.03 to about 1:3. In various embodiments, the molar ratio of deprotonated arsenous acid to the copper-based catalyst may range from about 1:0.03 to about 1:0.1, from about 1:0.1 to about 1:0.2, from about 1:0.2 to about 1:0.3, from about 1:0.3 to about 1:0.4, from about 1:0.4 to about 1:0.5, from about 1:0.5 to about 1:1, from about 1:1 to about 1:2, or from about 1:2 to about 1:3. In one exemplary embodiment, the mole:mole ratio of the deprotonated arsenous acid to copper-based catalyst may range from about 1:0.2 to about 1:0.3. In another exemplary embodiment, the copper-based catalyst is copper(II) sulfate, and the mole:mole ratio of the deprotonated arsenous acid to copper(II) sulfate may range from about 1:0.2 to about 1:0.3.

### (d) Step B—Reaction Conditions

In general, the reaction is conducted at a temperature that ranges from about 0° C. to about 120° C. In various embodiments, the reaction may be conducted at a temperature from about 0° C. to about 20° C., from about 20° C. to about 40° C., from about 40° C. to about 60° C., from about 60° C. to about 80° C., from about 80° C. to about 100° C., or from about 100° C. to about 120° C. In an exemplary embodiment, the temperature is from about 80° C. to about 100° C. The reaction generally is conducted in an inert atmosphere (e.g., under nitrogen or argon) and under ambient pressure.

In certain embodiments, the reaction may be allowed to proceed for about a period of time ranging from about 1 minute to about 5 minutes, from about 5 minutes to about 10 minutes, from about 10 minutes to about 15 minutes, from about 15 minutes to about 30 minutes, from about 30 minutes to about 1 hour, from about 1 hour to about 3 hours, from about 3 hours to about 6 hours. In an exemplary embodiment, the reaction is allowed to proceed for about 5 minutes to about 15 minutes.

In some embodiments, the arsenous acid reaction intermediate may be isolated from the reaction mixture using techniques known to those of skill in the art. Non-limiting examples of suitable techniques include precipitation, extraction, evaporation, distillation, chromatography, and crystallization. In other embodiments, the arsenous acid reaction intermediate may not be isolated and step (c) of the process may proceed in the same reaction pot or reactor.

The yield of the arsenous acid reaction intermediate can and will vary. Typically, the yield of the arsenous acid reaction intermediate may be at least about 40%. In one embodiment, the yield of the arsenous acid reaction intermediate may range from about 40% to about 60%. In another embodiment, the yield of the arsenous acid reaction intermediate may range from about 60% to about 80%. In a further embodiment, the yield of the arsenous acid reaction intermediate may range from about 80% to about 90%. In still another embodiment, the yield of the arsenous acid reaction intermediate may be greater than about 90%, or greater than about 95%.

### (e) Step C—Reaction Mixture

The method for producing a radiolabeled phenylarsonic acid further comprises contacting the solution from Step B with a phenyldiazonium salt to form a phenylarsonic acid. In the phenyldiazonium salt, the phenyldiazonium cation (also referred to as benzenediazonium) is paired with a counteranion, for example a halide, such as chloride, bromide or iodide; tetrafluoroborate, nitrate, sulfate, or phosphate. In an exemplary embodiment, the counteranion is tetrafluoroborate, thus forming the tetrafluoroborate salt. In some embodiments, the phenyldiazonium is optionally substituted, for example with an amino group or a lower alkane. When an amino-substituted phenyldiazonium salt is used, the reaction product is arsanilic acid, such as ortho-arsanilic acid, meta-arsanilic acid, or para-arsanilic acid.

The amount of phenyldiazonium salt added to the reaction mixture can and will vary. In general, the mole:mole ratio of the arsenous acid reaction intermediate to the phenyldiazonium salt may range from about 1:0.3 to about 1:30. In various embodiments, the molar ratio of arsenous acid reaction intermediate to the phenyldiazonium salt may range from about 1:0.3 to about 1:1, from about 1:1 to about 1:2, from about 1:2 to about 1:3, from about 1:3 to about 1:4, from about 1:4 to about 1:5, from about 1:5 to about 1:10, from about 1:10 to about 1:20, or from about 1:20 to about 1:30. In one exemplary embodiment, the mole:mole ratio of the arsenous acid reaction intermediate to phenyldiazonium salt may range from about 1:2 to about 1:4.

### (f) Step C—Reaction Conditions

In general, the reaction is conducted at a temperature that ranges from about −20° C. to about 40° C. In various embodiments, the reaction may be conducted at a temperature from about −20° C. to about −10° C., from about −10° C. to about 0° C., from about 0° C. to about 10° C., from about 10° C. to about 20° C., from about 20° C. to about 30° C., or from about 30° C. to about 40° C. In an exemplary embodiment, the temperature is about −10° C. to about 10° C., such as about −5° C. to about 5° C., or about 0° C. The reaction generally is conducted in an inert atmosphere (e.g., under nitrogen or argon) and under ambient pressure.

In general, the reaction may proceed for about 1 minute to about 12 hours. Typically, the duration of the reaction is longer at lower reaction temperatures. In certain embodiments, the reaction may be allowed to proceed for about a period of time ranging from about 1 minute to about 5 minutes, from about 5 minutes to about 10 minutes, from about 10 minutes to about 15 minutes, from about 15 minutes to about 30 minutes, from about 30 minutes to about 1 hour, from about 1 hour to about 3 hours, from about 3 hours to about 6 hours, from about 6 hours to about 9 hours, or from about 9 hours to about 12 hours.

In some embodiments, the phenylarsonic acid may be isolated from the reaction mixture using techniques known to those of skill in the art. Non-limiting examples of suitable techniques include precipitation, extraction, evaporation, distillation, chromatography, and crystallization. In an exemplary embodiment, the reaction mixture is neutralized and the phenylarsonic acid product is extracted.

The yield of the phenylarsonic acid can and will vary. Typically, the yield of the phenylarsonic acid may be at least about 40%. In one embodiment, the yield of the phenylarsonic acid may range from about 40% to about 60%. In another embodiment, the yield of the compound comprising phenylarsonic acid may range from about 60% to about 80%. In a further embodiment, the yield of the phenylarsonic acid may range from about 80% to about 90%. In still another embodiment, the yield of the phenylarsonic acid may be greater than about 90%, or greater than about 95%.

### (g) Sulfur-Containing Compounds

A radiolabeled phenylarsonic acid may be used to prepare radiopharmaceuticals, such as dithiophenylarsenic compounds. For example, phenyl groups stabilize arsenic dithiol compounds. Successful labeling of phenylarsenic acid with a radioisotope of arsenic advances the development of arsenic radiopharmaceuticals. Radiolabeled arsenic complexes may be formed between reaction of an arsenic acid, such as phenylarsonic acid or para-arsanilic acid, and a sulfur-containing compound selected from thioamine, monothiol, or dithiol. In an exemplary embodiment, the phenylarsonic acid is first reacted with at least two molar equivalents of a thioamine, followed by reaction with either at least two molar equivalents of a monothiol or at least one molar equivalent of a dithiol. The result of this reaction includes compounds having Formula (I), (II), or (III) as described above in section (I).

Non-limiting examples of thioamines include mercaptomethylamine, mercaptoethylamine, mercaptopropylamine, mercaptobutylamine, ammonium thioglycolate, cysteine, 2-aminoethanethiol (cysteamine), coenzyme A, and glutathione. Non-limiting examples of monothiols include methanethiol, ethanethiol, 1-propanethiol, 2-propanethiol, butanethiol, tert-butyl mercaptan, penanethiol, thiophenol, 2-mercaptoacetic acid, thioacetic acid, phenylmethanthiol (benzyl mercaptan), 2-mercaptoethanol, 3-mercaptopropane-1,2-diol, furan-2-ylmethanethiol, 2-mercaptoindole, and 2-(4-methylcyclohex-3-enyl)propane-2-thiol (thioterpineol). Non-limiting examples of dithiols include ethane-1,2-dithiol, 1,2-dimercaptosuccinic acid, 2,3-dimercaptosuccinic acid (DMSA), propane-1,3-dithiol, 6,8-dimercaptooctoic acid (thioctic acid), 1,5-dithiooctic acid, butane-1,4-dithiol, dithiothreitol, and dithioerythritol.

Non-limiting examples of the radiolabeled arsenic complexes include 1,2-dithioethanephenylarsine, 1,3-dithiopropanphenylarsine, (1,5-dithiooctic acid) phenylarsine, (1,2-dimercaptosuccinic acid) phenylarsine, 2-phenyl-1,3,2-dithiarsolane, 2-phenyl-1,3,2-dithiarsinane, 2-phenyl-1,3,2-dithiarsepane, 2-phenyl-1,3,2-dithiarsolane-4,5-dicarboxylic acid, 5-(2-phenyl-1,3,2-dithiarsinan-4-yl)pentanoic acid, 4-(1,3,2-dithiarsolan-2-yl)aniline, 2-(4-aminophenyl)-1,3,2-dithiarsolane-4,5-dicarboxylic acid, 4-(1,3,2-dithiarsinan-2-yl)aniline, and 5-(2-(4-aminophenyl)-1,3,2-dithioarsinan-4-yl)pentanoic acid.

An arsenic complex may be further derivatized with a biological vector, for example peptides such as bombesin or an antibody. Further examples of suitable biological vectors are described above in section (I)(b).

The amount of sulfur-containing reagent added to the reaction mixture can and will vary. In general, the mole:mole ratio of the phenylarsenic acid to the sulfur-containing reagent may range from about 1:0.3 to about 1:30. In various embodiments, the molar ratio of the phenylarsenic acid to the sulfur-containing reagent may range from about 1:0.3 to about 1:1, from about 1:1 to about 1:2, from about 1:2 to about 1:3, from about 1:3 to about 1:4, from about 1:4 to about 1:5, from about 1:5 to about 1:10, from about 1:10 to about 1:20, or from about 1:20 to about 1:30.

In some embodiments, the pH of the reaction mixture may be controlled to a pH of about 8 to about 5. In an exemplary embodiment, the pH may be set to about 6.

In general, the reaction may be conducted at a temperature that ranges from about 0° C. to about 120° C. In various embodiments, the reaction may be conducted at a temperature from about 0° C. to about 20° C., from about 20° C. to about 40° C., from about 40° C. to about 60° C., from about 60° C. to about 80° C., from about 80° C. to about 100° C., or from about 100° C. to about 120° C. In an exemplary embodiment, the reaction may be conducted at a temperature from about 50° C. to about 60° C. The reaction generally is conducted in an inert atmosphere (e.g., under nitrogen or argon) and under ambient pressure.

In general, the reaction may proceed for about 1 minute to about 12 hours. Typically, the duration of the reaction is longer at lower reaction temperatures. In certain embodiments, the reaction may be allowed to proceed for about a period of time ranging from about 1 minute to about 5 minutes, from about 5 minutes to about 10 minutes, from about 10 minutes to about 15 minutes, from about 15 minutes to about 30 minutes, from about 30 minutes to about 1 hour, from about 1 hour to about 3 hours, from about 3 hours to about 6 hours, from about 6 hours to about 9 hours, or from about 9 hours to about 12 hours. In an exemplary embodiment, the reaction may be allowed to proceed for about 0.5 hours to about 1.5 hours.

The yield of the arsenic complex can and will vary. Typically, the yield of the arsenic complex may be at least about 40%. In one embodiment, the yield of the arsenic complex may range from about 40% to about 60%. In another embodiment, the yield of the arsenic complex may range from about 60% to about 80%. In a further embodiment, the yield of arsenic complex may range from about 80% to about 90%. In still another embodiment, the yield of arsenic complex may be greater than about 90%, or greater than about 95%.

### DEFINITIONS

The compounds described herein have asymmetric centers. Compounds of the present invention containing an asymmetrically substituted atom may be isolated in optically active or racemic form. All chiral, diastereomeric, racemic forms and all geometric isomeric forms of a structure are intended, unless the specific stereochemistry or isomeric form is specifically indicated.

The term “acyl,” as used herein alone or as part of another group, denotes the moiety formed by removal of the hydroxly group from the group COOH of an organic carboxylic acid, e.g., RC(O)—, wherein R is R¹, R¹O—, R¹R²N—, or R¹S—, R¹ is hydrocarbyl, heterosubstituted hydrocarbyl, or heterocyclo, and R² is hydrogen, hydrocarbyl, or substituted hydrocarbyl.

The term “acyloxy,” as used herein alone or as part of another group, denotes an acyl group as described above bonded through an oxygen linkage (O), e.g., RC(O)O— wherein R is as defined in connection with the term “acyl.”

The term “alkyl” as used herein describes groups which are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

The term “alkenyl” as used herein describes groups which are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

The term “alkynyl” as used herein describes groups which are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The term “aromatic” as used herein alone or as part of another group denotes optionally substituted homo- or heterocyclic conjugated planar ring or ring system comprising delocalized electrons. These aromatic groups are preferably monocyclic (e.g., furan or benzene), bicyclic, or tricyclic groups containing from 5 to 14 atoms in the ring portion. The term “aromatic” encompasses “aryl” groups defined below.

The terms “aryl” or “Ar” as used herein alone or as part of another group denote optionally substituted homocyclic aromatic groups, preferably monocyclic or bicyclic groups containing from 6 to 10 carbons in the ring portion, such as phenyl, biphenyl, naphthyl, substituted phenyl, substituted biphenyl, or substituted naphthyl.

The terms “carbocyclo” or “carbocyclic” as used herein alone or as part of another group denote optionally substituted, aromatic or non-aromatic, homocyclic ring or ring system in which all of the atoms in the ring are carbon, with preferably 5 or 6 carbon atoms in each ring. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

The terms “halogen” or “halo” as used herein alone or as part of another group refer to chlorine, bromine, fluorine, and iodine.

The term “heteroatom” refers to atoms other than carbon and hydrogen.

The term “heteroaromatic” as used herein alone or as part of another group denotes optionally substituted aromatic groups having at least one heteroatom in at least one ring, and preferably 5 or 6 atoms in each ring. The heteroaromatic group preferably has 1 or 2 oxygen atoms and/or 1 to 4 nitrogen atoms in the ring, and is bonded to the remainder of the molecule through a carbon. Exemplary groups include furyl, benzofuryl, oxazolyl, isoxazolyl, oxadiazolyl, benzoxazolyl, benzoxadiazolyl, pyrrolyl, pyrazolyl, imidazolyl, triazolyl, tetrazolyl, pyridyl, pyrimidyl, pyrazinyl, pyridazinyl, indolyl, isoindolyl, indolizinyl, benzimidazolyl, indazolyl, benzotriazolyl, tetrazolopyridazinyl, carbazolyl, purinyl, quinolinyl, isoquinolinyl, imidazopyridyl, and the like. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

The terms “heterocyclo” or “heterocyclic” as used herein alone or as part of another group denote optionally substituted, fully saturated or unsaturated, monocyclic or bicyclic, aromatic or non-aromatic groups having at least one heteroatom in at least one ring, and preferably 5 or 6 atoms in each ring. The heterocyclo group preferably has 1 or 2 oxygen atoms and/or 1 to 4 nitrogen atoms in the ring, and is bonded to the remainder of the molecule through a carbon or heteroatom. Exemplary heterocyclo groups include heteroaromatics as described above. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The term “oxygen-protecting group” as used herein denotes a group capable of protecting an oxygen atom (and hence, forming a protected hydroxyl group), wherein the protecting group may be removed, subsequent to the reaction for which protection is employed, without disturbing the remainder of the molecule. Exemplary oxygen protecting groups include ethers (e.g., allyl, triphenylmethyl (trityl or Tr), p-methoxybenzyl (PMB), p-methoxyphenyl (PMP)), acetals (e.g., methoxymethyl (MOM), β-methoxyethoxymethyl (MEM), tetrahydropyranyl (THP), ethoxy ethyl (EE), methylthiomethyl (MTM), 2-methoxy-2-propyl (MOP), 2-trimethylsilylethoxymethyl (SEM)), esters (e.g., benzoate (Bz), allyl carbonate, 2,2,2-trichloroethyl carbonate (Troc), 2-trimethylsilylethyl carbonate), silyl ethers (e.g., trimethylsilyl (TMS), triethylsilyl (TES), triisopropylsilyl (TIPS), triphenylsilyl (TPS), t-butyldimethylsilyl (TBDMS), t-butyldiphenylsilyl (TBDPS) and the like. A variety of oxygen protecting groups and the synthesis thereof may be found in “Protective Groups in Organic Synthesis” by T. W. Greene and P. G. M. Wuts, 3^{rd} ed., John Wiley & Sons, 1999.

The “substituted hydrocarbyl” moieties described herein are hydrocarbyl moieties which are substituted with at least one atom other than carbon, including moieties in which a carbon chain atom is substituted with a heteroatom such as nitrogen, oxygen, silicon, phosphorous, boron, or a halogen atom, and moieties in which the carbon chain comprises additional substituents. These substituents include alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

Having described the invention in detail, it will be apparent that modifications and variations are possible without departing from the scope of the invention defined in the appended claims.

### EXAMPLES

The following examples are included to demonstrate certain embodiments of the invention. It should be appreciated by those of skill in the art that the techniques disclosed in the examples represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and scope of the invention, therefore all matter set forth is to be interpreted as illustrative and not in a limiting sense.

### Example 1

### Phenyldiazonium Tetrafluoroborate—Method A

Sodium tetrafluoroborate (NaBF₄, 6.7 g, 61 mmol) was added to a 100-mL round-bottomed flask in an ice-water bath. Deionized water (18Ω, 30 mL) and concentrated hydrochloric acid (HCl, 16 mL) were added. The solution was cooled and rapidly stirred. Aniline (5 mL, 55 mmol) was added and the solution cooled for 30 minutes. Meanwhile, a solution of sodium nitrite (NaNO₂) was prepared by dissolving 3.8 g (55 mmol) in 6 mL of water and cooled on an ice-water bath. The nitrile solution was added dropwise to the round-bottomed flask over a period of 20 minutes. The solution was allowed to react for about 5 minutes, resulting in a nearly white precipitant. The precipitant was filtered and washed sequentially with 95% ethanol and diethyl ether, yielding 5.7 grams (55%). The product was stored in a freezer until use.

### Example 2

### Phenyldiazonium Tetrafluoroborate—Method B

A solution of sodium nitrite (0.833 g, 1.67 mL) was slowly added to a stirring mixture of aniline (1.0 g) and 48% fluoroboric acid (HBF₄, 5.55 mL) cooled to 0° C. Following addition of the sodium nitrite, the reaction mixture was stirred for 10 more minutes and then filtered. The precipitant was washed sequentially with 48% fluoroboric acid, 95% ethanol, and a copious amount of diethyl ether. The initial yield was 61%, and additional product was obtained from the filtrate. The combined product was stored in a desiccator until use.

### Example 3

### Phenylarsonic Acid

At a temperature of about 50° C., a supersaturated stock solution (2.83 M) of sodium carbonate (Na₂CO₃) was prepared in deuterium oxide (D₂O). A stock of copper(II) sulfate (CuSO₄) solution (1.21 M) was also prepared in D₂O.

In a vessel 0.200 mL of the Na₂CO₃ solution (0.566 mmol) was combined with 0.300 mL of D₂O and 22.3 mg of arsenic trioxide (As₂O₃, 16.3 mg of As, 225 μmol) and heated to about 90° C. with stirring until the arsenic trioxide dissolved. The CuSO₄ solution (66 μL, 80 μmol was added. The combined solution was allowed to stir at 90° C. for about 10 minutes. The solution was transferred to an ice-water bath and allowed to equilibrate to a temperature of 0° C. A solution of phenyldiazonium tetrafluoroborate (127.9 mg, 666 μmol, a mole:mole ratio of 3:1 diazonium/arsenic) in 1.5 mL D₂O was also prepared and cooled to 0° C. with stirring. The solution was filtered and a 0.500 mL D₂O rinse of reaction flask was also passed through the filter. The filter was washed with D₂O (twice with 0.300 mL and once with 0.200 mL). Concentrated HCl (94 μL, 1.14 mmol) was added to the filtrate to neutralize the carbonate. The solution was then filtered through a 0.22-μm filter. A clear light yellow precipitate formed after filtration. The phenylarsonic acid was identified through ¹H-NMR and mass spectrometry.

### Example 4

### Radiolabeled ⁷⁶As-Phenylarsonic Acid

Arsenic-76 was obtained from the University of Missouri Research Reactor and behaved analogously to arsenic-72 and arsenic-77. About 2.35 mg of ⁷⁶As-labeled As₂O₃ (1.78 mg As, 23.8 μmol) in 0.1 mL of aqueous 2 M sodium carbonate (Na₂CO₃) was added to 0.5 mL of aqueous 2 M Na₂CO₃ in a two-necked round-bottomed flask (1200 μmol total). This solution was heated in an oil bath at 80° C. to ensure complete dissolution of arsenic trioxide. A solution of aqueous copper(II) sulfate (CuSO₄, 37 μL, 1.61 M, 60 μmol) was added to the flask and the solution was allowed to cool to room temperature before it was transferred to an ice bath. A slurry of 0.1035 g of phenydiazonium tetrafluoroborate (0.540 mmol) in 1 mL water and 0.25 mL ethanol also at 0° C. was added to the flask in five equal portions of 0.25 mL each at four-minute intervals. An additional 0.25 mL water was also added after four more minutes. The green solution turned dark brown upon the first addition of the diazonium salt. After the additions, the solution was filtered to remove a brown precipitate. The filtrate was neutralized with 0.15 mL concentrated hydrochloric acid (HCl) and filtered through a 0.22-micron filter. The phenylarsenous acid was observed by high-pressure liquid chromatography (HPLC) with ultraviolet (UV) detection at 254 nm. A radiation detector at the same elution time showed a clear signal for the ⁷⁶As analog. A yield of about 18% was observed for the ⁷⁶As-phenylarsonic acid product.

### Example 5

### Radiolabeled ⁷⁷As-Phenylarsonic Acid and ⁷⁷As-Dithioglycolylphenylarsine

No-carrier-added arsenic, as ⁷⁷As-arsenate, in methanol was separated from a neutron-irradiated, enriched ⁷⁶GeO₂ target. The target had been dissolved in 2 M KOH, neutralized with concentrated HCl, diluted in methanol, and passed through three methanol prewetted Waters CE Plus Accell short Sep-pak™ cartridges (360 mg each) arranged in tandem and fitted with a 0.2-μm Whatman filter disc, and eluted with 10 mL of methanol. The methanol was removed under a gentle stream of air. Deionized water (18Ω, 0.9 mL) and ammonium thioglycolate (5.5 M, 0.1 mL) were added to the residue, which was then heated to 60° C. for 50 minutes while stirring. Phenyldiazonium tetrafluoroborate (153.0 mg, 79.7 mmol) was added as a solid, vortexed for 30 seconds, and then ethyl ether (2 mL) and water (18Ω, 1 mL) were added. The aqueous phase was separated, filtered through a 0.2-μm Whatman filter disc prior to injection onto an HPLC for analysis.

An aliquot (0.2 mL) was analyzed by reversed phase HPLC on a Phenomenex Luna Phenyl-Hexyl column (3p, 100×4.6 mm) using a Shimadzu HPLC system with UV-vis detection at 254 nm and 280 nm, and radioactivity detection on a Beckman 170 radioisotope detector. A gradient elution system at a flow rate of 0.5 mL/min was used (3 minutes at pH 2.75 phosphate buffer, a linear gradient to 70/30 pH 2.75 phosphate buffer/acetonitrile over 8 minutes, to 40/60 over 9 minutes, and finally to 20/80 over 5 minutes). The ⁷⁷As-phenylarsonic acid labeled preparation described above eluted with a retention time of 13.89 minutes, while the non-radioactive standard compound eluted at 13.63 minutes. This 0.25-minute time difference is attributed to the time between the two detectors. An additional ⁷⁷As-labeled compound was observed with a retention time of approximately 33 minutes. The identity of this second species may be dithioglycolylphenylarsine the desired phenyl arsenic acid with two thioglycolic acid groups replacing the two hydroxyl groups or possibly the diphenylarsonic acid analogue.

What is claimed is:

1. A composition comprising a compound of Formula (I): 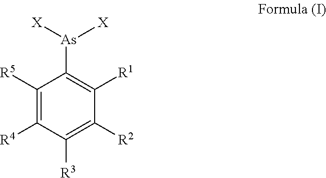
wherein arsenic is radioactive;
R¹, R², R³, R⁴, and R⁵ are independently selected from hydrogen, hydrocarbyl, substituted hydrocarbyl, amines, substituted amines, halogens, hydroxyl, thiol, or nitro groups or are joined together to form one or more fused ring systems selected from hydrocarbyl, substituted hydrocarbyl, aromatic, and heteroaromatic rings;
X represents ligand atoms wherein X is chosen from carbon, sulfur, and nitrogen; and
X may be further substituted by hydrogen, hydrocarbyl or substituted hydrocarbyl.

2. The composition of claim 1, wherein the arsenic is chosen from ⁷²As or ⁷⁷As.

3. The composition of claim 1, wherein R³ is NH₂.

4. The composition of claim 1, wherein R³ is NH₂; and R¹, R², R⁴, and R⁵ are each hydrogen.

5. The composition of claim 1, wherein each X is sulfur and is substituted by hydrogen, hydrocarbyl or substituted hydrocarbyl.

6. The composition of claim 1, wherein one or both X are substituted by carboxylic acids, phenyl groups or amines.

7. The composition of claim 1, wherein one or both X are substituted by CH₂COOH.

8. The composition of claim 5, wherein one or both X are substituted by CH₂Ph.

9. The composition of claim 5, wherein one or both X are substituted by CH₂CH₂NH₂.

10. The composition of claim 5, wherein the composition is isolated.

11. A composition comprising Formula (II): 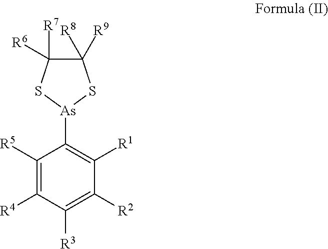
wherein arsenic is radioactive;
R¹, R², R³, R⁴, and R⁵ are independently selected from hydrogen, hydrocarbyl, substituted hydrocarbyl, amines, substituted amines, halogens, hydroxyl, thiol, or nitro groups or are joined together to form one or more fused ring systems selected from hydrocarbyl, substituted hydrocarbyl, aromatic and heteroaromatic rings; and
R⁶, R⁷, R⁸, and R⁹ are independently selected from hydrogen, hydrocarbyl, substituted hydrocarbyl, amines, substituted amines, halogens, hydroxyl, thiol, or nitro groups or are joined together to form one or more fused ring systems selected from hydrocarbyl and substituted hydrocarbyl rings and aromatic and heteroaromatic rings.

12. The composition of claim 11, wherein the arsenic is chosen from ⁷²As or ⁷⁷As.

13. The composition of claim 11, wherein R³ is NH₂; and R¹, R², R⁴, and R⁵ are each hydrogen.

14. The composition of claim 11, wherein R⁶, R⁷, R⁸, and R⁹ are each hydrogen.

15. The composition of claim 11, wherein one or more of R⁶, R⁷, R⁸, and R⁹ are substituted with a C₁-C₁₀ carboxylic acid.

16. The composition of claim 11, wherein R⁶ and R⁸ are substituted by COOH.

17. The composition of claim 16, wherein R⁷ and R⁹ are substituted by hydrogen.

18. The composition of claim 11, where one of R⁶, R⁷, R⁸, or R⁹ is substituted by a linear C₁-C₅ carboxylic acid.

19. The composition of claim 11, wherein R⁶, R⁷, R⁸, or R⁹ are substituted such to form a fused ring system with one or more hydrocarbyl, substituted hydrocarbyl, aromatic or heteroaromatic rings.

20. The composition of claim 11, wherein the composition is isolated.

21. A composition comprising Formula (III): 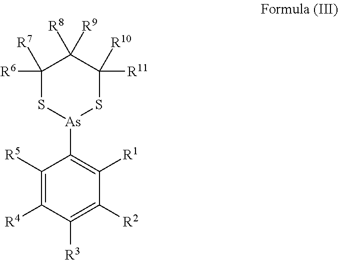
wherein arsenic is radioactive;
R¹, R², R³, R⁴, and R⁵ are independently selected from hydrogen, hydrocarbyl, substituted hydrocarbyl, amines, substituted amines, halogens, hydroxyl, thiol, or nitro groups or are joined together to form one or more fused ring systems selected from hydrocarbyl, substituted hydrocarbyl rings, aromatic and heteroaromatic rings; and
R⁶, R⁷, R⁸, R⁹, R¹⁰, and R¹¹ are independently selected from hydrogen, hydrocarbyl, substituted hydrocarbyl, amines, substituted amines, halogens, hydroxyl, thiol, or nitro groups or are joined together to form one or more fused ring systems selected from hydrocarbyl, substituted hydrocarbyl, aromatic and heteroaromatic rings.

22. The composition of claim 21, wherein the arsenic is chosen from ⁷²As or ⁷⁷As.

23. The composition of claim 21, wherein R³ is NH₂ and R¹, R², R⁴, and R⁵ are each hydrogen.

24. The composition of claim 21, wherein R⁶, R⁷, R⁸, R⁹, R¹⁰, and R¹¹ are each hydrogen.

25. The composition of claim 21, wherein one or more of R⁶, R⁷, R⁸, R⁹, R¹⁰, and R¹¹ are substituted with a C₁-C₁₀ carboxylic acid.

26. The composition of claim 21, wherein one or more of R⁶, R⁸, or R¹⁰ are substituted by COOH.

27. The composition of claim 21, where one of R⁶, R⁷, R⁸, R⁹, R¹⁰, and R¹¹ is substituted by a C₁-C₅ carboxylic acid.

28. The composition of claim 21, wherein two or more of R⁶, R⁷, R⁸, R⁹, R¹⁰, and R¹¹ are substituted such to form a fused ring system with one or more hydrocarbyl, substituted hydrocarbyl, aromatic or heteroaromatic ring systems.

29. The composition of claim 21, wherein the composition is isolated.

30. A radiopharmaceutical comprising Formula (I), (II), or (III): 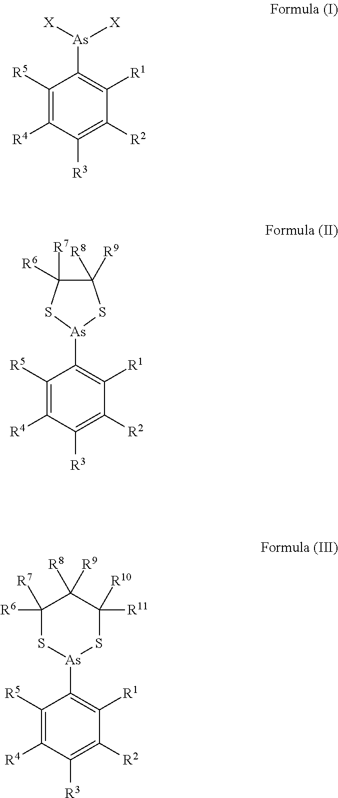
wherein arsenic is radioactive;
X represents ligand atoms wherein X is chosen from carbon, sulfur, and nitrogen;
X may be further substituted with hydrogen, hydrocarbyl or substituted hydrocarbyl;
R¹, R², R³, R⁴, and R⁵ are independently selected from hydrogen, hydrocarbyl, substituted hydrocarbyl, amines, substituted amines, halogens, hydroxyl, thiol, or nitro groups or are joined together to form one or more fused ring systems selected from hydrocarbyl, substituted hydrocarbyl, aromatic and heteroaromatic rings; and
R⁶, R⁷, R⁸, R⁹, R¹⁰, and R¹¹ are independently selected from hydrogen, hydrocarbyl, substituted hydrocarbyl, amines, substituted amines, halogens, hydroxyl, thiol, or nitro groups or are joined together to form one or more fused ring systems selected from hydrocarbyl and substituted hydrocarbyl rings and aromatic and heteroaromatic rings.

31. The radiopharmaceutical of claim 30, wherein the arsenic is chosen from ⁷²As or ⁷⁷As.

32. The radiopharmaceutical of claim 30, wherein R³ is NH₂; and R¹, R², R⁴, and R⁵ are each hydrogen.

33. The radiopharmaceutical of claim 30, wherein R⁶, R⁷, R⁸, R⁹, R¹⁰, and R¹¹ are independently selected from hydrogen, hydrocarbyl, substituted hydrocarbyl.

34. The radiopharmaceutical of claim 30, wherein R⁶, R⁷, R⁸, R⁹, R¹⁰, and R¹¹ are independently selected from hydrogen, carboxylic acid, and C₁-C₁₀ carboxylic acids.

35. The radiopharmaceutical of any one of claim 30, wherein the compound of Formula (I), (II), or (III) is conjugated to a peptide.

36. The radiopharmaceutical of claim 35, wherein the peptide is bombesin.

37. The radiopharmaceutical of claim 35, wherein the peptide is a site-specific antibody.

38. A method of producing radiolabeled phenylarsonic acid, the method comprising:
(a) contacting a radiolabeled arsenous acid with a carbonate to form a first solution;
(b) optionally contacting the first solution with a copper-based catalyst to form a second solution; and
(c) contacting the second solution with a phenyldiazonium salt to give a third solution comprising the radiolabeled phenylarsonic acid.

39. The method of claim 38, wherein the radiolabeled arsenous acid is chosen from As₂O₃ or H₃AsO₃.

40. The method of claim 38, wherein the carbonate is sodium carbonate.

41. The method of claim 38, wherein the phenyldiazonium salt is tetrafluoroborate salt.

42. The method of claim 38, wherein the first solution is heated to about 70° C. to about 90° C.

43. The method of claim 38, wherein the copper-based catalyst is selected from the group consisting of copper(I) bromide (CuBr), copper(I) cyanide (CuCN), copper(I) chloride (CuCl), copper(I) fluoride (CuF), copper(I) iodide (CuI), copper chromite (Cu₂Cr₂O₅), copper(I) oxide (Cu₂O), copper(I) sulfide (Cu₂S), copper(I) phosphate (Cu₃(PO₄)₂), copper(I) phosphide (Cu₃P), copper tetrafluoroborate (Cu(BF₄)₂), copper(II) bromide (CuBr₂), copper(II) carbide (CuC₂), copper(II) cyanide (Cu(CN)₂), copper(II) carbonate (CuCO₃), copper(II) chloride (CuCl₂), copper(II) fluoride (CuF₂), copper(II) nitrate (Cu(NO₃)₂), copper(II) oxide (CuO), copper(II) hydroxide (Cu(OH)₂), copper(II) iodide (CuI₂), copper(II) sulfide (CuS), copper(II) sulfate (CuSO₄), copper(II) arsenate (Cu₃(AsO₄)₂), potassium hexafluorocuprate (K₃CuF₆), copper(III) oxide (Cu₂O₃), and cesium hexafluorocuprate (Cs₂CuF₆).

44. The method of claim 43, wherein the copper-based catalyst is CuSO₄; and the mole to mole ratio of arsenous acid to CuSO₄ ranges from about 1:2 to 1:3 arsenous acid to CuSO₄.

45. The method of claim 38, wherein step (c) is conducted at about 0° C.

46. The method of claim 38, wherein the process further comprises neutralizing and filtering the third solution.

47. The method of claim 38, wherein the radiolabeled phenylarsonic acid is isolated.

48. The method of claim 38, wherein the radiolabeled phenylarsonic acid is further reacted with a thioamine and a monothiol or dithiol.
